SEQUENCE LISTING (17 sequences)

SEQ ID NO: 1 - protein, 120 aa, Toxoplasma gondii
MASVKRVVVAVMIVNVLALIFVGVAGSTRDVGSGGDDSEGARGREQQQVQQHEQNEDRSLFERGRAAVTGHPVRTAVGLAAAVVAVVSLLRLLKRRRRRAIQEESKESATAEEEEVAEEE

SEQ ID NO: 2 - DNA, 363 bp, Toxoplasma gondii
atggcgtctgtaaaacgcgtcgttgtggcggtaatgatcgtgaacgtgctggctttaatttttgtgggcgttgccggttcaacgcgtgacgtagggtcaggcggggatgactccgaaggtgctagggggcgtgaacaacaacaggtacaacaacacgaacaaaatgaagaccgatcgttattcgaaaggggaagagcagcggtgactggacatccagtgaggactgcagtgggacttgctgcagctgtggtggccgttgtgtcactactgcgattgttgaaaaggaggagaagacgcgcgattcaagaagagagcaaggagtctgcaaccgcggaagaggaagaagttgccgaggaagagtaa

SEQ ID NO: 3 - protein, 50 aa, Artificial
  Amino acids 26-75 of the sequence of GRA5 protein from RH strain of toxoplasma gondii
GSTRDVGSGGDDSEGARGREQQQVQQHEQNEDRSLFERGRAAVTGHPVRT

SEQ ID NO: 4 - protein, 50 aa, Artificial
  Amino acids 26-75 of the sequence of GRA5 protein from PRU strain of toxoplasma gondii
GSTRDTGSGGDDSEGAWGGEQQQVQQHGQSEDRSLFERGRAAVTGHPVRT

SEQ ID NO: 5 - protein, 50 aa, Artificial
  Amino acids 26-75 of the sequence of GRA5 protein from 76K strain of toxoplasma gondii
GSTCDTGSGGDDSEGAWGGEQQQVQQHGQSEDRSLFERGRAAVTGHPVRT

SEQ ID NO: 6 - protein, 50 aa, Artificial
  Amino acids 26-75 of the sequence of GRA5 protein from C56 and CEP strains of toxoplasma gondii
GSTRDVGSGADDSEGAGGRERQQVQQHEQNEDRSLFERGRAAVTGHPVRT

SEQ ID NO: 7 - protein, 220 aa, Artificial
  glutathione S-transferase protein
SPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDL

SEQ ID NO: 8 - DNA, 31 bp, Artificial
  Primer creating a XhoI restriction site
tcaactcgagccgcgtcggtttggtttgtgc

SEQ ID NO: 9 - DNA, 31 bp, Artificial
  Primer creating a XhOI restriction site
gtatctcgaggggcagacgtggccggtttcc

SEQ ID NO: 10 - DNA, 27 bp, Artificial
  Primer creating a SmaI restriction site
gtcccgggggttcaacgcgtgacgtag

SEQ ID NO: 11 - DNA, 27 bp, Artificial
  Primer creating a SmaI restriction site
gtcccgggggttcaacgtgtgacacag

SEQ ID NO: 12 - DNA, 25 bp, Artificial
  Primer creating an EcoRI restriction site
ccgaattccctcactggatgtccag

SEQ ID NO: 13 - protein, 283 aa, Artificial
  Fusion protein of glutathione S-transferase and amino acids 26-75 of the sequence of GRA5 protein from RH strain of toxoplasma gondii
SPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLIEGRGIPGGSTRDVGSGGDDSEGARGREQQQVQQHEQNEDRSLFERGRAAVTGHPVREFIVTD

SEQ ID NO: 14 - protein, 283 aa, Artificial
  Fusion protein of glutathione S-transferase and amino acids 26-75 of the sequence of GRA5 protein from 76K strain of toxoplasma gondii
SPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLIEGRGIPGGSTCDTGSGGDDSEGAWGGEQQQVQQHGQSEDRSLFERGRAAVTGHPVREFIVTD

SEQ ID NO: 15 - protein, 283 aa, Artificial
  Fusion protein of glutathione S-transferase and amino acids 26-75 of the sequence of GRA5 protein from C56 and CEP strains of toxoplasma gondii
SPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLIEGRGIPGGSTRDVGSGADDSEGAGGRERQQVQQHEQNEDRSLFERGRAAVTGHPVREFIVTD

SEQ ID NO: 16 - protein, 29 aa, Artificial
  Amino acids 30-58 of the sequence of GRA5 protein from PRU strain of toxoplasma gondii
DTGSGGDDSEGAWGGEQQQVQQHGQSEDR

SEQ ID NO: 17 - protein, 27 aa, Artificial
  Amino acids 37-63 of the sequence of GRA5 protein from 76K strain of toxoplasma gondii
DSEGAWGGEQQQVQQHGQSEDRSLFER

(19) United States
(12) Patent Application Publication
(10) Pub. No.: US 2010/0160219 A1
(43) Pub. Date: Jun. 24, 2010

(54) Novel Polypeptides Inducing Dendritic Cell Migration, as Well as Medicines and Pharmaceutical Compositions Containing Same

(76) Inventors: Florence Persat, Bron (FR); Claude Vincent, Loyon (FR); Marie-France Cesbron-Delauw, La Tronche (FR)

Correspondence Address: CLARK & BRODY, 1700 Diagonal Road, Suite 510, Alexandria, VA, 22314, US

(21) Appl. No.: 12/226,744

(22) PCT Filed: Apr. 27, 2007
(86) PCT No.: PCT/FR2007/000728

§ 371 Date: Oct. 27, 2009

(30) Foreign Application Priority Data
Apr. 21, 2006 (DE) 10 2006 019 178.1

(51) Int. Cl. A61K 38/16; C07K 14/00; C07H 21/00; A61P 37/08; A61P 37/00

(52) U.S. Cl. 514/12; 530/324; 536/23.1; 530/350

(57) ABSTRACT

The invention concerns a polypeptide corresponding to amino acids 26 to 75 of the sequence of GRA5-RH protein of SEQ ID No 1: MASVKRVWAVMIVNVLALIFVGVAGSTRDVGSGGDDSEGARGRE QQQVQQHEQNEDRSLFERGRAAVT-GHPVRTAVGLAAAWAWSLL RLLKRRRRRAIQEESKESATAEEEEVAEEE, its variants acting on dendritic cell migration, homologues acting on dendritic cell migration, and fragments thereof acting on dendritic cell migration, as well as the pharmaceutical compositions thereof comprising such polypeptides and their use for making a medicine for preventing or treating skin and mucous membrane conditions involving dendritic cells or Langerhans cells.

The present invention relates to the technical field of the treatment of allergic and autoimmune diseases. In particular, the present invention relates to novel polypeptides that induce the migration of dendritic cells, nucleic acids encoding such polypeptides, and medicinal products and pharmaceutical compositions containing such polypeptides.

Toxoplasma gondii is a protozoan parasite that causes toxoplasmosis. It has been demonstrated that under certain conditions Toxoplasma gondii parasites, regardless of which strain is used, secrete excreted-secreted antigens, some of which, called GRAs, come from the dense granules. The GRA1, GRA2, GRA5, and GRA6 genes, which encode the excreted-secreted antigens of Toxoplasma gondii, are described in the following publications:

GRA1 (P23): CESBRON-DELAUW et al. (Proc. Natl. Acad. Sci. 1989, 86, p. 7537-7541) WO89/05658

GRA2 (GP28.5): FR 2692282, FR 2702491; MERCIER et al., Mol. Biochem. Parasitol, 1993, 58, 71-82)

GRA5 (P21): LECORDIER et al. (Mol. Biochem. Parasitol., 1993, 59, 143-154); FR 2702491. The GRA5 protein (or P21, 21 kDa) is characterised by a signal peptide, two N- and C-terminal hydrophilic regions flanking a central hydrophobic domain (Lecordier et al., Mol. Biochem. Parasitol 1993, 59, 143-154). The amino acid sequence of the GRA5 protein (120 aa) exhibits no similarity to any proteins of known function. In order to determine the function of GRA5, a toxoplasma mutant has been isolated in which the GRA5 gene is disrupted (called GRA5 KO). The intracellular growth of GRA5 KO parasites is identical to that of the wild type RH strain. Their virulence in mice does not appear altered (Mercier et al., Mol. Biochem. Parasitol. 2001, 116, 247-251). The GRA5 protein secreted by the tachyzoites of the RH strain has the sequence

SEQ ID NO. 1:
MASVKRVVVAVMIVNVLALIFVGVAGSTRDVGSGGDDSEGARGRE
QQQVQQHEQNEDRSLFERGRAAVTGHPVRTAVGLAAAVVAVVSLL
RLLKRRRRRAIQEESKESATAEEEEVAEEE

(deposited in the UniProtKB/Swiss-Prot gene bank, accession number AC Q07828) and was subsequently named GRA5—RH.

The DNA sequence

SEQ ID NO. 2:
ATGGCGTCTGTAAAACGCGTCGTTGTGGCGGTAATGATCGTGAACGTGCT
GGCTTTAATTTTTGTGGGCGTTGCCGGTTCAACGCGTGACGTAGGGTCAG
GCGGGGATGACTCCGAAGGTGCTAGGGGGCGTGAACAACAACAGGTACAA
CAACACGAACAAAATGAAGACCGATCGTTATTCGAAAGGGGAAGAGCAGC
GGTGACTGGACATCCAGTGAGGACTGCAGTGGGACTTGCTGCAGCTGTGG
TGGCCGTTGTGTCACTACTGCGATTGTTGAAAAGGAGGAGAAGACGCGCG
ATTCAAGAAGAGAGCAAGGAGTCTGCAACCGCGGAAGAGGAAGAAGTTGC
CGAGGAAGAGTAA

encoding GRA5-RH is listed in GenBank with the accession number: L06091.

GRA6 (P32): FR 2702491.

The GRA proteins form a family of molecules of low molecular weight (between 20 and 40 kDa). These proteins are stored in the dense granules of toxoplasma and are secreted in the parasitophorous vacuole. To date, 9 proteins have been characterised (GRA1 through GRA9) from the dense granules of the tachyzoite form (Mercier et al., Int. J. Parasitol., 2005, 35, 829-849). The biological stimulus that induces the exocytosis of the GRA proteins into the vacuolar compartment once the parasites are established in the host cell is currently unknown. However, massive discharge of the contents of the dense granules can be induced under cell-free conditions, after incubating the parasites in serum-supplemented medium (ESA) (Darcy et al., Parasite Immunol. 1988, 10, 553-567). Under these conditions, the quantity of GRA proteins secreted is dependent on the concentration of serum proteins (10 to 40 μg/mg of total protein/hour for 10 to 40% serum) (Coppens et al., Eur. J. Cell Biol. 1999, 78, 463-472). Under these conditions, the GRA proteins are found in the medium essentially in a soluble form (Lecordier et al., Mol. Biol. Cell 1999, 10, 1277-1287).

Various studies have been conducted by the inventors (Diana et al., Clinical and Experimental immunology, 2005, 1-9 and FEMS Immunology and Medical Microbiology, 2004, 42, 321-331) on the supernatant secreted by various strains of Toxoplasma gondii, which have demonstrated that the latter affect the migration and maturation of dendritic cells in vitro.

Within the context of the invention, the inventors have demonstrated that the migration-inducing activity resides in a polypeptide corresponding to the N-terminal portion of the GRA5 protein and that this polypeptide could have the capacity to deplete a tissue of dendritic cells.

In particular, the subject matter of the present invention is the polypeptide corresponding to amino acids 26 to 75 of the sequence of the GRA5-RH protein of

SEQ ID NO. 1:
MASVKRVVVAVMIVNVLALIFVGVAGSTRDVGSGGDDSEGARGRE
QQQVQQHEQNEDRSLFERGRAAVTGHPVRTAVGLAAAVVAVVSLL
RLLKRRRRRAIQEESKESATAEEEEVAEEE

variants and homologues thereof, as well as fragments thereof, with activity on the migration of dendritic cells.

Amino acids 26 to 75 of the sequence of the GRA5-RH protein correspond to the sequence

SEQ ID NO. 3:
GSTRDVGSGG DDSEGARGRE QQQVQQHEQN EDRSLFERGR
AAVTGHPVRT

As well as the polypeptide of SEQ ID NO.3, the invention pertains to variants, homologues and fragments of this polypeptide, as well as fragments of said variants and homologues, where the said fragments, homologues and variants have activity on the migration of dendritic cells.

In particular, the polypeptides of the invention are selected from the polypeptides of sequence:

SEQ ID NO. 3:
GSTRDVGSGG DDSEGARGRE QQQVQQHEQN EDRSLFERGR
AAVTGHPVRT
SEQ ID NO. 4:
GSTRDTGSGG DDSEGAWGGE QQQVQQHGQS EDRSLFERGR
AAVTGHPVRT
SEQ ID NO. 5:
GSTCDTGSGG DDSEGAWGGE QQQVQQHGQS EDRSLFERGR
AAVTGHPVRT
SEQ ID NO. 6:
GSTRDVGSGA DDSEGAGGRE RQQVQQHEQN EDRSLFERGR
AAVTGHPVRT

homologues or fragments thereof, with activity on the migration of dendritic cells.

Preferably, the polypeptides of the invention are selected from the polypeptides of sequence SEQ ID NO.3, SEQ ID NO.4, SEQ ID NO.5 and SEQ ID NO.6 and the polypeptides of sequence SEQ ID NO.3, SEQ ID NO.4, SEQ ID NO.5 and SEQ ID NO.6 in which the C-terminal amino acid (threonine) has been deleted.

As used in the description of the invention, the terms below are defined as follows.

The terms “polypeptide” or “protein” are used interchangeably within the invention to refer to a chain of amino acids; no lower or upper size limit is associated with either term.

“GRA5-RH protein” refers to the GRA protein which has a molecular weight of about 21 KDa that is secreted by Toxoplasma gondii tachyzoites of the RH strain. This secretion can notably take place under the conditions described by Darcy et al. (cited above). The GRA5 protein secreted by the tachyzoites of the RH strain has the sequence

SEQ ID NO. 1:
MASVKRVVVAVMIVNVLALIFVGVAGSTRDVGSGGDDSEGARGRE
QQQVQQHEQNEDRSLFERGRAAVTGHPVRTAVGLAAAVVAVVSLL
RLLKRRRRRAIQEESKESATAEEEEVAEEE

and is called GRA5-RH. The DNA sequence

SEQ ID NO. 2:
ATGGCGTCTGTAAAACGCGTCGTTGTGGCGGTAATGATCGTGAACGTGCT
GGCTTTAATTTTTGTGGGCGTTGCCGGTTCAACGCGTGACGTAGGGTCAG
GCGGGGATGACTCCGAAGGTGCTAGGGGGCGTGAACAACAACAGGTACAA
CAACACGAACAAAATGAAGACCGATCGTTATTCGAAAGGGGAAGAGCAGC
GGTGACTGGACATCCAGTGAGGACTGCAGTGGGACTTGCTGCAGCTGTGG
TGGCCGTTGTGTCACTACTGCGATTGTTGAAAAGGAGGAGAAGACGCGCG
ATTCAAGAAGAGAGCAAGGAGTCTGCAACCGCGGAAGAGGAAGAAGTTGC
CGAGGAAGAGTAA

that encodes GRA5-RH is listed in GenBank with the accession number: L06091.

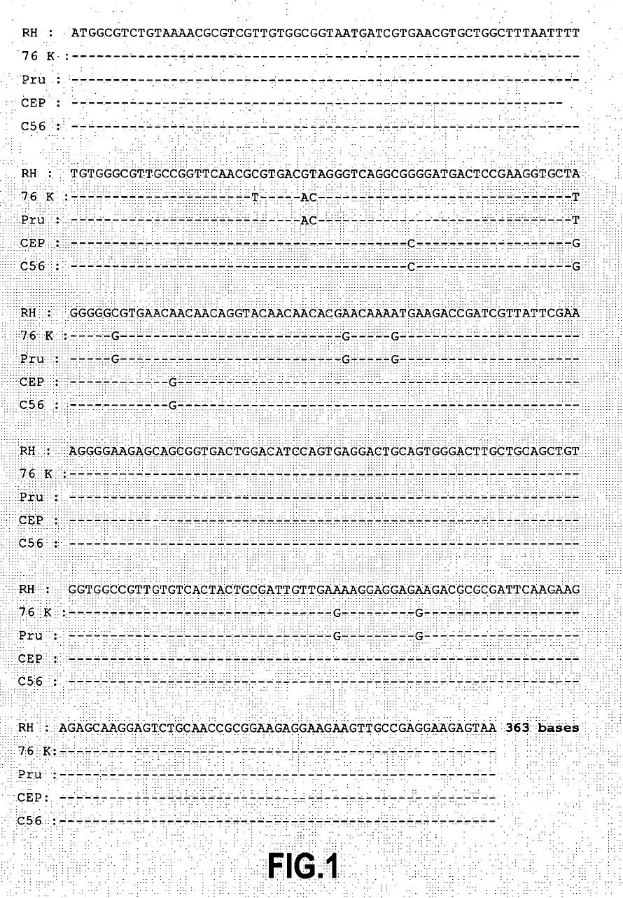

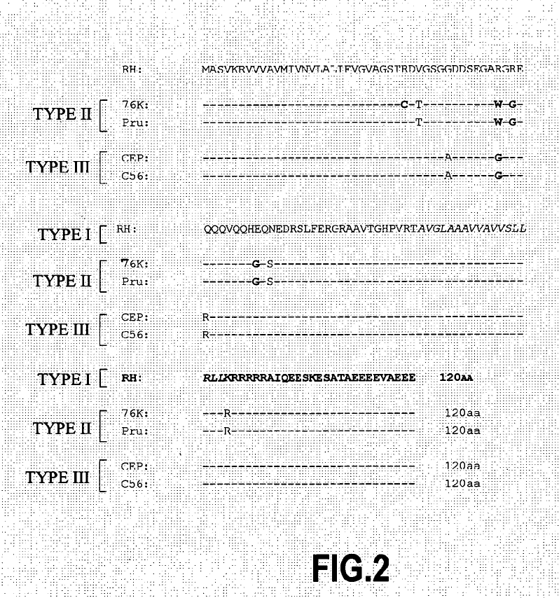

The expression “variants of the GRA5-RH protein” refers to all the allelic variants of the GRA5-RH protein, i.e. all the GRA5 proteins secreted by Toxoplasma gondii tachyzoites other than those of the RH strain, for example under the conditions described by Darcy et al. (cited above) or by the inventors (Diana et al., 2005, cited above). These GRA5 proteins usually differ from the GRA5 protein secreted by the RH strain by at least one deletion, addition or substitution of at least one amino acid, a truncation, en elongation and/or chimeric fusion. It can also be considered that these variants bind to the monoclonal antibody TG 17-113 (Charif, H., et al. Exp. Parasitol. 1993, 71, 114-124). Tachyzoites derived from various strains can secrete such GRA5 proteins. Toxoplasma gondii parasites of different genotypes (I, II or III) secrete this protein. FIGS. 1 and 2 show the gene of the GRA5 protein in different Toxoplasma gondii strains (RH deposited in the ATCC under accession no. 50174, 76K described by Laugier M., et al. Ann. Parasitol. Hum. Comp. 1970, 45, 389-403, Pru described by Martrou P. et al. Limousin Medical. 1965, 53, 3-7, CEP deposited in the ATCC under accession no. 50842 and C56 deposited in the ATCC under accession no. 50951).

Variants of the polypeptide corresponding to amino acids 26 to 75 of the sequence of a GRA5-RH protein correspond to a fragment of a GRA5 protein, secreted by tachyzoites derived from a Toxoplasma gondii strain other than the RH strain, the secretion occurring, for example, under the conditions described by Darcy et al. (cited above). For strains other than the RH strain, positions 26 to 75 are determined after alignment with the GRA5-RH sequence of SEQ ID NO.1, and correspond to the amino acids of the sequence situated from position 26 to position 75 of SEQ ID NO.1, as illustrated in FIG. 2. Therefore, the polypeptides corresponding to amino acids 26 to 75 of the sequence of a GRA5 protein have the following sequences in the following strains:

RH STRAIN
SEQ ID NO. 3:
GSTRDVGSGG DDSEGARGRE QQQVQQHEQN EDRSLFERGR
AAVTGHPVRT
PRU STRAIN
SEQ ID NO. 4:
GSTRDTGSGG DDSEGAWGGE QQQVQQHGQS EDRSLFERGR
AAVTGHPVRT
76K STRAIN
SEQ ID NO. 5:
GSTCDTGSGG DDSEGAWGGE QQQVQQHGQS EDRSLFERGR
AAVTGHPVRT
C56 STRAIN AND CEP STRAIN
SEQ ID NO. 6:
GSTRDVGSGA DDSEGAGGRE RQQVQQHEQN EDRSLFERGR
AAVTGHPVRT

The polypeptides of SEQ ID NO. 4, SEQ ID NO. 5 and SEQ ID NO. 6 are examples of variants of the polypeptide of SEQ ID NO.3.

A polypeptide “homologue” is understood to mean a polypeptide which, relative to the polypeptide of which it is a homologue, exhibits notably at least one deletion, addition or substitution of at least one amino acid, a truncation, an elongation and/or chimeric fusion.

Preferred polypeptide homologues and variants are those which have an amino acid sequence exhibiting at least 80% homology, more preferably at least 85%, at least 87%, at least 90%, at least 93%, at least 95%, at least 97%, at least 98%, at least 99% and most preferably 100% homology with the amino acid sequences of the polypeptides of which they are variants or homologues. Preferably, the polypeptide homologues or variants only differ from the reference polypeptide by one, two or three amino acid changes, said changes being selected from additions, deletions or substitutions. In the case of a substitution, one or more consecutive or non-consecutive amino acids can be replaced by equivalent amino acids. The expression equivalent amino acid is understood to mean any amino acid that may replace one of the amino acids of the basic structure, but without modifying their characteristics or essential functional properties, such as their activity on the migration of dendritic cells. The groups of amino acids below are generally recognised as being equivalents and correspond to conservative substitutions that result in a protein of equivalent shape and function:
Ala, Ser, Thr, Pro, Gly
Asn, Asp, Glu, Gln,
His, Arg, Lys,
Met, Leu, Ile, Val,
Phe, Tyr, Trp.

The invention also pertains to fragments, with activity on the migration of dendritic cells, of the polypeptide of SEQ ID NO.3, as well as active fragments of a homologue or variant of the polypeptide of SEQ ID NO.3. Such fragments consist of a minimum of 15 consecutive amino acids, and preferably 17, 20, 23, 25 or 30 consecutive amino-acids. Examples of such fragments are the polypeptides of sequence SEQ ID NO.3, SEQ ID NO.4, SEQ ID NO.5 and SEQ ID NO.6 in which the C-terminal amino acid (threonine) has been deleted.

Fragments of the polypeptide of the invention obtained by cleaving said polypeptide using a proteolytic enzyme or a chemical reagent, or by placing said polypeptide in a strongly acidic medium also form part of the invention.

Other examples of fragments are peptides corresponding to amino acids 30 to 58, or 37 to 63 of the sequence of a GRA5 protein, and in particular of the sequence of a GRA5 protein from the RH, PRU, 76K, CR6 or CEP strains.

In the case of the PRU strain, the fragment corresponding to amino acids 30 to 58 of the GRA5-PRU protein exhibits the sequence:

SEQ ID NO. 16: DTGSGGDDSEGAWGGEQQQVQQHGQSEDR

the fragment corresponding to amino acids 37 to 63 of the GRA5-PRU protein exhibits the sequence:

SEQ ID No. 17: DSEGAWGGEQQQVQQHGQSEDRSLFER

A “fusion protein” of two polypeptides is understood to refer to co-linear linkage of two polypeptides which can potentially be connected by a linker arm, preferably comprising 2 to 6 amino acids.

The present invention also relates to fusion proteins between GST and a polypeptide as defined previously, as well as homologues or fragments of such proteins, with activity on the migration of dendritic cells.

Such fusion proteins are, for example, the protein of sequence

SEQ ID NO. 13:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G RG I
P G G S T R D V G S G G D D S E G A R G R E Q Q Q
V Q Q H E Q N E D R S L F E R G R A A V T G H P V
R E F I V T D

the protein of sequence

SEQ ID NO. 14:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G RG I
P G G S T C D T G S G G D D S E G A W G G E Q Q Q
V Q Q H E Q S E D R S L F E R G R A A V T G H P V
R E F I V T D

the protein of sequence

SEQ ID NO. 15:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G RG I
P G G S T R D V G S G A D D S E G A G G R E R Q Q
V Q Q H E Q N E D R S L F E R G R A A V T G H P V
R E F I V T D

The sequence corresponding to GST can be cleaved off by incubating the above-mentioned proteins with a protease called factor Xa.

The expression “with activity on the migration of dendritic cells” is understood to mean a polypeptide that induces the migration of dendritic cells, determined by means of the in vitro assay detailed in the example below, corresponding to at least 70%, preferably to at least 80%, at least 90%, and most preferably to at least 95% of the activity of the polypeptide of SEQ ID NO.3. The migration assay involves preliminary incubation of dendritic cells with the polypeptide or fraction to be tested, and 24 hours later the cells are washed and loaded onto the upper part of a migration chamber, the lower part containing the chemokine MIP3-β. The cells that have migrated from the upper compartment to the lower compartment are counted, and migration is quantified by determining the percentage they represent of the number of cells that were initially loaded into the upper chamber. The activity of a polypeptide corresponds to the percentage of cells that have migrated. For the purpose of comparing different experiments and in order to be able to analyse the data statistically, the percentage migration results are normalised by calculating a migration index with reference to the spontaneous migration of dendritic cells (migration index).

Preferably, a polypeptide of the invention is a polypeptide composed of the sequence SEQ ID NO.3, SEQ ID NO.4, SEQ ID NO.5, SEQ ID NO.6, SEQ ID NO.16 or SEQ ID NO.17, or of a sequence with at least 80% homology, preferably at least 85%, at least 90%, at least 95%, at least 98% and most preferably at least 99% homology with SEQ ID NO.3, SEQ ID NO.4, SEQ ID NO.5, SEQ ID NO.6, SEQ ID NO.16 or SEQ ID NO.17, following optimal alignment. The expression “polypeptide with an amino acid sequence exhibiting a percentage homology of at least 80%, preferably at least 85%, at least 90%, at least 95%, at least 98% and most preferably at least 99% after optimal alignment with a reference sequence” is understood to refer to polypeptides exhibiting certain modifications relative to the reference polypeptide, such as in particular one or more deletions, truncations, an elongation, chimeric fusion, and/or one or more substitutions.

The percentage homology between two amino acid sequences is understood in the present invention to refer to the percentage of amino acid residues that are similar (identical or conservative substitution) or, preferably, identical between the two sequences to be compared, obtained after optimal alignment of the two sequences, where the differences between the two sequences may be distributed randomly and anywhere along the length of the sequence. The term “optimal alignment” is understood to refer to the alignment that produces the highest percentage homology, determined as described below. The optimal alignment, corresponding to the alignment that gives the highest number of identical amino acids following alignment of the two amino acid sequences being compared, may potentially be obtained by sliding the sequences relative to one another and/or inserting a gap between 2 consecutive amino acids in one of the sequences being compared.

The degree of homology, as understood in the invention, can be determined locally using techniques for comparing protein sequences, and for example the BLAST P technique as described by ALTSCHUL S. F. et al. in Nucleic Acid Research 1997, 25, 3389-3402. In this case, a protein is considered to be a homologue of another reference protein sequence when it exhibits, on a number of amino acids at least equal to 80% of the number of amino acids of the reference protein sequence, preferably along the entire length of the reference sequence, percentage homology corresponding to the values indicated previously, namely at least 80% homology, preferably at least 85%, at least 90%, at least 95%, at least 98% and most preferably at least 99% homology. The percentage homology can be calculated using given (sic) by the BLAST P technique version 2.2.13. Preferably, the comparison is made on the entire sequence of the polypeptide to be compared and the complete sequence of the reference polypeptide and the degree of homology of at least 80%, preferably at least 85%, at least 90%, at least 95%, at least 98% and most preferably at least 99% homology, must be obtained by comparing the entire protein sequence to be compared and the complete sequence of the reference protein.

Preferably, the percentage homology is calculated, manually, over the entire length of the polypeptide of which the degree of homology with a reference polypeptide is sought. The percentage homology is then calculated, after optimal alignment, by counting the number of positions for which the polypeptides exhibit identical amino acids and by dividing this number of identical positions by the length of the sequence of the longest polypeptide and multiplying the result obtained by 100 to obtain the percentage homology between these two sequences.

The present invention also pertains to any purified or isolated nucleic acid formed by a sequence of nucleotides encoding the polypeptides of the invention. They may be sequences of natural or artificial origin, and in particular genomic DNA, cDNA, mRNA, hybrid sequences or synthetic or semi-synthetic sequences. They can be obtained by any technique known to the skilled person, and in particular through screening libraries, chemical synthesis or combined methods, including chemical or enzymatic modification of sequences obtained by screening libraries.

The terms nucleic acid, nucleic sequence or nucleic acid sequence, polynucleotide, oligonucleotide, polynucleotide sequence and nucleotide sequence, which will be used interchangeably in the present description, are understood to refer to a precise sequence of modified or unmodified nucleotides, which can be used to define a fragment or a region of a nucleic acid, which may or may not contain non-natural nucleotides, and which could equally be double-stranded DNA, single-stranded DNA or the transcription products of said DNA molecules, and/or an RNA fragment.

The nucleic acids of the invention can be prepared notably by chemical synthesis and genetic engineering using techniques well known to the skilled person and described, for example, in SAMBROOK et al. “Molecular Cloning: a Laboratory Manual” published in 1989 by Cold Spring Harbor Press, NY, second edition.

For example, the synthesis of the cDNA sequences of the invention can be performed by amplification of mRNA species using the PCR method (Polymerase Chain Reaction), as described, for example, by GOBLET et al. (Nucleic Acid Research, 17, 2144, 1989) using synthetic oligonucleotides as primers, determined from the DNA sequence of GRA5.

The amplified nucleic acid fragment may then be cloned using the techniques described in AUSUBEL et al. (Current Protocols in Molecular Biology, Green Publishing Associates and Wiley Interscience, New-York, 1989, updated until 1997, chapter 3).

The proteins and peptide fragments of the invention can be obtained by the technique of genetic engineering that involves the following steps:
culture of a transformed microorganism or eukaryotic or prokaryotic cells transformed with an expression vector containing a sequence of nucleic acids of the invention; and
recovery of the protein or peptide fragment produced by said microorganism or said eukaryotic cells.

This technique is well known to the skilled person. More details about, the technique can be found in the book: Recombinant DNA Technology I, Editors Ales Prokop, Raskesh K Bajpai; Annals of the New-York Academy of Sciences, volume 646, 1991.

They can also be prepared using conventional peptide synthesis, which is well known to the skilled person.

The invention also pertains to prokaryotic microorganisms and eukaryotic cells transformed using an expression vector containing a DNA sequence of the invention. As well as the DNA sequence of the invention, this expression vector, which may for example take the form of a plasmid, cosmid or phage, must include the means necessary for its expression, such as notably a promoter, a transcription termination signal, a replication origin and preferably a selection marker. The transformation of microorganisms and eukaryotic cells is a technique well known to the skilled person, who, depending on the microorganism to be transformed, could easily determine the necessary means for expression of the DNA sequence of the invention.

An example of a prokaryotic microorganism is E. coli and an example of a eukaryote is the yeast Saccharomyces cerevisiae.

Notable examples of eukaryotic, cells that are suitable for the purposes of the invention are COS monkey cells, CHO Chinese hamster ovary cells, Sf9 insect cells, the Jurkat human T cell lymphoma line, etc., all of which are listed in the ATCC.

Preferably, a system that employs a vector of the baculovirus type can be used. This system enables the production of proteins that are foreign to the cells (Sf9 cells) of an insect (Spodoptera frugiperda) using in vivo recombination between a transfer vector (plasmid) that contains firstly the foreign DNA sequence and secondly the genome of a virus. (O'Reilly, D., L. K. Miller, and V. A. Luckow. 1992. Baculovirus expression vectors: A Laboratory manual. W.H. Freeman and Company, New York, Y.).

The invention also pertains to prokaryotic microorganisms and eukaryotic cells co-transformed with expression vectors containing the DNA sequence encoding a polypeptide of the invention, where the said expression vectors also contain the means for their expression, including in the yeast two-hybrid system.

The polypeptides of the invention are particularly interesting therapeutically. Their therapeutic activity is particularly related to their ability to cause the migration of dendritic cells from tissues or mucosae, which has been demonstrated in vitro and in vivo in mice. They will be useful in particular for the treatment of diseases where it is necessary to deplete a peripheral tissue, such as the skin or mucosae, of dendritic cells.

The invention also relates to the polypeptides and fusion proteins of the invention for their application as medicinal products, as well as pharmaceutical compositions containing one of the polypeptides or one of the fusion proteins of the invention, combined with at least one suitable pharmaceutical excipient. The polypeptides of the invention can also be used in fusion, for example, with another protein such as GST of sequence

SEQ ID NO. 7:
S P I L G Y W K I K G L V Q P T R L L L E Y L
E E K Y E E H L Y E R D E G D K W R N K K F E L G
L E F P N L P Y Y I D G D V K L T Q S M A I I R Y
I A D K H N M L G G C P K E R A E I S M L E G A V
L D I R Y G V S R I A Y S K D F E T L K V D F L S
K L P E M L K M F E D R L C H K T Y L N G D H V T
H P D F M L Y D A L D V V L Y M D P M C L D A F P
K L V C F K K R I E A I P Q I D K Y L K S S K Y I
A W P L Q G W Q A T F G G G D H P
P K S D L

These pharmaceutical compositions are prepared using conventional techniques well known to the skilled person. The pharmaceutical excipients are selected according to the desired pharmaceutical form and administration method, for example, oral, sublingual, subcutaneous, intramuscular, intravenous or topical administration, etc. Compositions with a form suitable for topical application, for example cream or ointment, are preferred.

The present invention also relates to the use of a polypeptide or fusion protein of the invention for the preparation of a medicinal product intended to prevent or treat diseases of the skin or mucosae, where dendritic cells or Langerhans cells are implicated. In particular, a polypeptide of the invention can be used to manufacture a medicinal product intended to prevent or treat diseases selected from: atopic dermatitis, inflammations, autoimmune diseases, systemic lupus erythematosus, polyarthritis, psoriasis, graft versus host disease, graft rejection, or allergies, such as contact allergies, for example to latex, cosmetics, perfumes, tattoos, metals and their salts, pulmonary allergic phenomena due to airborne allergens, allergic rhinitis, asthma or food allergies. Depending on how it is used and on the target dendritic cells used, the administration of a polypeptide of the invention may also lead to a heightened immune response.

The invention will now be described in detail by means of the experimental overview below.

A large proportion of the techniques described in these examples, which are well known to the skilled person, are presented in detail in the book by SAMBROOK et al. (cited above) or in the book by AUSUBEL et al. (cited above).

The description below will be understood better by referring to FIG. 1 through, 9 on which:

FIG. 1 represents alignment of the DNA sequences encoding the GRA5 protein from different strains of Toxoplasma gondii,

FIG. 2 represents alignment of the peptide sequences of the GRA5 protein from different strains of Toxoplasma gondii,

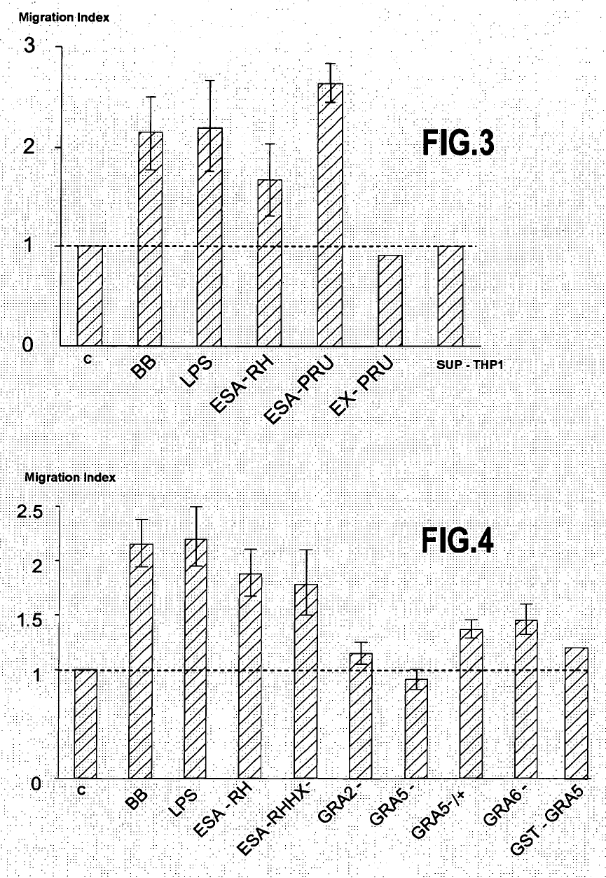

FIG. 3 through 7 illustrate the migration indexes obtained in the various assays performed.

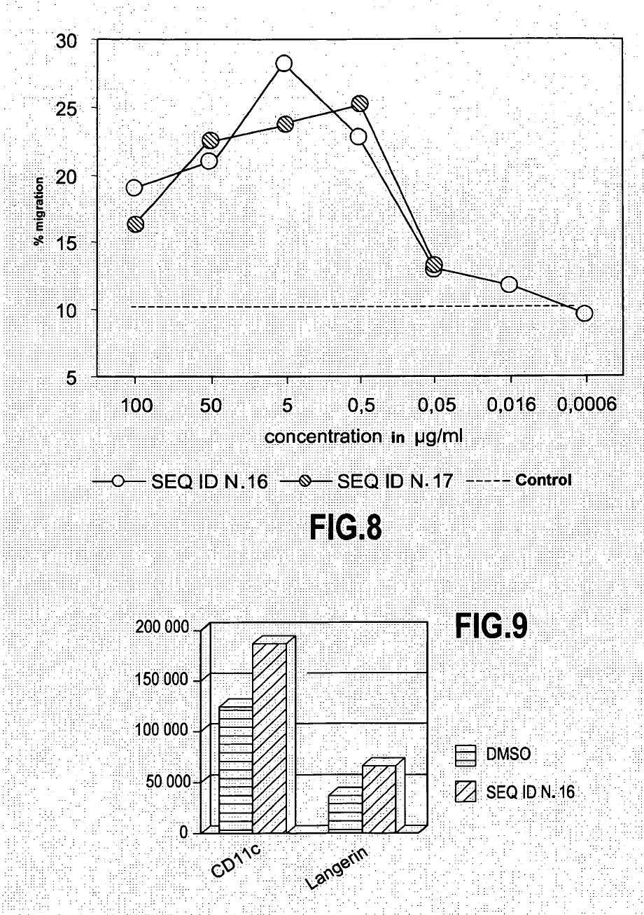

FIG. 8 presents the in vitro migration-inducing activity of increasing concentrations of the peptide of SEQ ID NO.16 and of the peptide of SEQ ID NO.17.

FIG. 9 shows the number of Langerhans cells and dendritic cells identified by labelling with an anti-CD11c antibody, obtained on an in vitro (sic) assay in mouse.

### EXAMPLES

### Reagents Used

RPMI 1640 culture medium with Glutamax (ref 61870-010, In Vitrogen Gibco, Grand Island, N.Y., USA)

Foetal calf serum (FCS, ref 16000-036, In Vitrogen Gibco, Grand Island, N.Y., USA), inactivated by heat treatment at 56° C. for 30 minutes

Bovine serum albumin (ref 652 237, 100 mg/ml, Roche Diagnostics GmbH, Mannheim, FRG)

2-mercaptoethanol (ref 31 350-010, 50 mM, In Vitrogen Gibco, Grand Island, N.Y., USA)

Gentamicin (ref 15 710-049, 10 mg/ml, In Vitrogen Gibco, Grand Island, N.Y., USA)

Penicillin-streptomycin (ref 15140-122 penicillin: 10,000 U/ml, streptomycin: 10,000 U/ml In Vitrogen Gibco Grand Island, N.Y., USA)

GM-CSF (recombinant granulocyte macrophage colony-stimulating factor; ref 11343128, 1 mg, 11×10⁶ U/mg, AL-Immunotools, Friesoythe, Germany)

TNF-α (recombinant human tumour necrosis factor; specific activity 2×10⁷ U/ml, 200 μg/ml; Genzyme Corp., Boston, Mass., USA) Boehringer

TGF-β1 (transforming growth factor beta 1, 10 μg; ref 100B010; R&D Systems, Minneapolis; MN, USA)

MIP-3β (macrophage inflammatory protein 3β∀., ref 361-MI, 25 μg/ml; R&D Systems, Minneapolis, Minn., USA).

Trypan blue (ref T8154, 0.4%; Sigma-Aldrich, Saint-Louis, Mo., USA)

Matrigel GFR (Matrigel Growth Factor Reduced, ref 354263, 5 ml; BD Biosciences, San Jose, Calif., USA).

6-well culture plate (ref [35]3502, Falcon)

12-well culture plate (ref [35]3503, Falcon)

24-well culture plate (ref [35]3504, Falcon)

Insert for 6-well plates, pore size 8 μm (ref [35]3093, Falcon)

Insert for 12-well plates, pore size 8 μm (ref [35]3082, Falcon)

Insert for 24-well plates, pore size 8 μm (ref [35]3097, Falcon)

5-ml round bottom culture tubes (ref [35]2054, Falcon)

Disposable Kova slide counting chambers (ref 87144, Hycor Biomedical LTD, Penicuik, United Kingdom)

Restriction enzymes and modification enzymes (Boehringer-Mannheim, Mannheim, Germany).

T4 DNA ligase and Taq-Polymerase (Promega, Charbonnières, France).

Bacteria strain BL21 (Stratagene).

Plasmid vectors: pPCR Amp Script SK(+) (cloning kit, Stratagene, La Jolla, Calif.); pGEX-3X (Pharmacia, Uppsala, Sweden)

Agarose beads coupled to Glutathione and reduced glutathione (SIGMA Chimie, St-Quentin, France).

Bradford Kit (Bio-Rad)

“Deaza G/A^{T7} Sequencing™ Mixes” kit (Pharmacia)

“AutoRead™ Sequencing Kit” (Pharmacia).

Oligonucleotides

| | | | Restriction |
|---|---|---|---|
| Primer | Sequence | Sense^{a} | site created |
| 21.19 | SEQ ID No. 8 | S | XhoI |
| | 5′- | | |
| | TCAACTCGAGCCGCGTCGGTTTGGTTTGTGC- | | |
| | 3′ | | |
| 21.20 | SEQ ID No. 9 | AS | XhoI |
| | 5′- | | |
| | GTATCTCGAGGGGCAGACGTGGCCGGTTTCC- | | |
| | 3′ | | |
| G5N5′ | SEQ ID No. 10 | S | SmaI |
| | 5′-GTCCCGGGGGTTCAACGCGTGACGTAG-3′ | | |
| G5N5′76K | SEQ ID No. 11 | S | SmaI |
| | 5′-GTCCCGGGGGTTCAACGTGTGACACAG-3′ | | |
| G5N3′ | SEQ ID No. 12 | AS | EcoRI |
| | 5′-CCGAATTCCCTCACTGGATGTCCAG-3′ | | |

^{a}5′-sense, AS: anti-sense

### Solutions Used:

RPMI Medium with 5% FCS or 10% FCS

Add 25 or 50 ml of FCS to one 500-ml bottle of medium. On the day of use, sterilely remove the required volume, add 10 μl of gentamicin per ml of medium and bring to room temperature before using.

RPMI Medium/1% BSA

Add 1 ml of BSA to 99 ml of RPMI in a 100-ml bottle. On the day of use, sterilely remove the required volume, add 10 μl of gentamicin per ml of medium and bring to room temperature before using.

Matrigel Solution

Prepare the Matrigel at 4° C. on ice with RPMI medium at 4° C. The Matrigel is diluted with RPMI medium to obtain a concentration of 8.35 mg/ml, and is then divided into 100 μl aliquots and stored at −20° C.

RPMI Medium/Chemokine

Add 100 μl of MIP3β to 5 ml of RPMI/1% BSA, homogenise using a vortex mixer. This solution is sufficient to prepare 2 wells of a 6-well plate, 5 wells of a 12-well plate, 12 wells of a 24-well plate.

GM-CSF Solution

A stock solution is prepared by adding 5.5 ml of sterile water to 1 mg of GM-CSF; this solution has a concentration of 2×10⁶ U/ml. It is divided into aliquots of 500 μl and is stored at −80° C.
A secondary solution is prepared from the previous solution by the addition of one 500-μl tube to 4.5 ml of RPMI/5% FCS. This solution is in turn divided into aliquots of 100 μl and stored at −80° C.
Thawed tubes can be kept for 8 days at 4° C., and 1 μl of this solution is used per ml of culture medium.

TNF-α Solution

The TNFα is divided into 100 μl aliquots and stored at −80° C. A stock solution is prepared by diluting one 100-μl tube by the addition of 700 μl of RPMI/10% FCS. This solution is stored in aliquots of 100 μl at −80° C.
A secondary solution is prepared from the previous solution by addition of one 100-μl tube to 0.9 ml of RPMI/5% FCS. This solution is in turn divided into aliquots of 15 and 30 μl and stored at −80° C.
Thawed tubes can be kept for 8 days at 4° C., and 1 μl of this solution is used per ml of culture medium.

Preparation of the TGF-β1

The 10 μg contained in the vial are suspended in 5 ml of RPMI/10% FCS in the presence of 20 μl of HCl 1N. This stock solution is stored in aliquots of 250 μl at −80° C.
An intermediate solution is prepared by diluting one 250-μl tube in 750 μl of RPMI/10% FCS. This solution is divided into aliquots of 15 μl and 30 μl and stored at −80° C.
Thawed tubes can be kept for 8 days at 4° C., and 1 μl of this solution is used per ml of culture medium.

### Stability of Reagents and Solutions

The FCS, the Matrigel and the cytokines are stored frozen at −80° C. indefinitely.

The culture medium, the bovine serum albumin, the 2-mercaptoethanol, the gentamicin and the Trypan blue are stored in the refrigerator until their expiry date.

Serum-supplemented media are stored in the refrigerator and used within one month. The intermediate solutions of cytokine and Matrigel are divided into aliquots then stored at −80° C.

### Culture of Tachyzoites of the RH Strain on the THP1 Cell Line

1. The THP1 cells are washed in RPMI (10 min at 1400 rpm) then counted in the presence of Trypan blue (v/v).
2. Place 10×10⁶ THP1 cells and 10×10⁶ tachyzoites in a 25 cm² flask. Culture in 10 ml of RPMI/5% FCS with 1% penicillin-streptomycin.
3. The cells can be frozen after 24 hours. Alternatively they are transferred into a 75 cm² flask, adding 10 ml of medium.
4. After 48 hours the cells should be completely lysed, and the tachyzoites are recovered.
5. Recover the tachyzoites using a 25 ml pipette, dissociating clumps by drawing them up into the pipette then expelling them with the tip of the pipette held against the bottom of the flask. Lyse the remaining THP1 cells by transferring them with an insulin syringe fitted with its needle. Transfer to 50 ml tubes (40 ml per tube).
6. Centrifuge at low speed (4 min at 700 rpm), and recover the supernatant which contains the tachyzoites.
7. If the quantity of debris or intact THP1 cells is too great, filter the supernatant with a 8 μM filter then a 3 μM filter to separate the tachyzoites from the surviving THP1 cells.
8. Dilute in PBS and centrifuge for 20 min at 1400 rpm.
9. Resuspend the pellet in 10 ml of PBS, and mix on a vortex mixer at a setting not exceeding 1500.
10. Count the tachyzoites.

### Culture of Tachyzoites of the PRU Strain on the THP1 Cell Line

1. Place 10×10⁶ THP1 cells and 10×10⁶ tachyzoites in a 25 cm² flask. Culture in 10 ml of RPMI/5% FCS with 1% penicillin-streptomycin.
2. After 24 hours, transfer to a 75 cm² flask, adding 10 ml of medium.
3. Change the culture medium every 3-4 days by centrifugation at 1400 rpm for 10 min. Split the flask if the THP1 cells have multiplied and if the medium is yellow.
4. Periodically check the level of infection by staining the THP1 cells with acridine orange and ethidium bromide (5 μg/ml of each). When the percentage of infected cells has reached 30%, it is possible to freeze it.
5. Continue culturing until a maximum level of free tachyzoites and complete cell lysis have been obtained.
6. Recover the tachyzoites with a 10 ml pipette and transfer them to 50-ml tubes (40 ml per tube).
7. Centrifuge at low speed (4 min at 700 rpm), and recover the supernatant containing the tachyzoites.
8. If the quantity of debris or intact THP1 cells is too great, filter the supernatant with a 8 μM filter then a 3 μM filter to separate the tachyzoites from the surviving THP1 cells. Run 2 ml of PBS through the filter beforehand, followed by the tachyzoites; do not use the plunger but allow the suspension to run through the filter by gravity alone.
9. Dilute in PBS and centrifuge for 20 min at 1400 rpm.
10. Resuspend the pellet in 10 ml of PBS, and mix on a vortex mixer at a setting not exceeding 1800.
11. Count the tachyzoites.

### Preparation of the Secreted Antigens (ESA)

ESAs are obtained from tachyzoites of different strains. Tachyzoites from the following are sources are recovered after passaging on THP1 cells: mouse ascites (virulent RH strain); mouse brain (less virulent Pru strain); parasitised cells frozen in liquid nitrogen.
1. Centrifuge the tachyzoites for 20 min at 1400 rpm
2. Resuspend the pellet in RPMI medium with 10% FCS or 2 mg/ml of purified human albumin to a concentration of 150×10⁶ tachyzoites per ml.
3. Transfer into either a well of a 6-well plate or into a culture tube. Incubate at 37° C. for 3 hours. Agitate the plate every 15 min; tubes are placed on a Dynal rotating mixer in the incubator.
4. After 3 hours, transfer into an Eppendorf tube and centrifuge for 15 min at 1000 rpm.
5. Recover the supernatant, filter through a 0.22μ filter and freeze in liquid nitrogen.
6. Recover the pellet and check that the parasites are still alive.

### Preparation of Genomic DNA

Genomic DNA was prepared from the strains of T. gondii (RH, Pru, 76K, CEP and C56) using tachyzoites derived from the ascites of infested mice or from infection of a layer of Hep-II cells. The parasites were collected then purified after filtration on a polycarbonate membrane with a pore size of 3 μm (Nucléopore). After centrifugation for 10 minutes at 3000 rpm, the parasites were washed twice in PBS. The pellet was resuspended in a solution of 0.1 M EDTA, 10 mM Tris-HCl pH 8.0, 1% SDS and 2 μg/ml proteinase K, and incubated overnight at 50° C. Contaminating proteins were removed by phenol/chloroform extraction. The DNA was precipitated with two volumes of absolute ethanol then washed in 70% ethanol. The DNA was solubilised in TE buffer (10 mM Tris-HCl, 1 mM EDTA, pH 8.0) overnight at 4° C.; its concentration was deduced after measuring the optical density of the solution at 260 nm.

### Gene Amplification (PCR)

PCR amplification reactions were performed under standard conditions using Peltier Thermal Cycler apparatus (PTC-200, MJ Research) with 5 to 10 ng of plasmid template or 1 μg of genomic DNA, in a reaction volume of 50 μl. The templates were placed in the presence of 0.5 μM oligonucleotide primers, a mixture of deoxyribonucleotides (dATP, dCTP, dGTP, dTTP, 200 μM, Pharmacia) and 4 units of Deep Vent DNA polymerase. The cycles are 94° C.-5 min (94° C. 1 min-60° C. 1 min-72° C. 2 min) 35 times, with a final extension at 72° C. for 7 min.

### Sequencing

Manual sequencing was performed in the presence of [³⁵S] α-dATP (Amersham) using the “Deaza G/A^{T7} Sequencing™ Mixes” kit (Pharmacia). The sequencing reactions were analysed after electrophoretic migration on a 6% polyacrylamide gel in 1×TBE buffer. The gel was then fixed for 15 minutes in a solution of 10% acetic acid, 10% methanol, then dried and visualised by autoradiography (Biomax film, Kodak).

Automatic sequencing was performed in the presence of fluorochrome-labelled dATP (Cy™5-13-dATP) using the AutoRead™ Sequencing Kit (Pharmacia).

### Production of Recombinant Glutathione S-Transferase (GST) Fusion Proteins

The fusion polypeptides or the wild type GST protein were produced in accordance with the procedures recommended by Pharmacia. Briefly, cultured recombinant BL21 cells in exponential phase were used, protein expression was induced for 3 hours at 37° C. by the addition of 1 mM isopropyl-β-D-thiogalactoside (IPTG, Roche). The cells are centrifuged at 4000×g for 15 min and the pellet is resuspended in lysis buffer (0.02 M phosphate pH 7.4, 0.5 mM phenyl methyl sulfonyl fluoride, 1 mM EDTA, 1% Triton X100). The cells are lysed on ice by two consecutive passes through a French press (1000 psi). The lysate is centrifuged at 10,000×g at 4° C. for 10 min. The fusion polypeptides are purified from the supernatant by affinity chromatography using glutathione immobilised onto agarose beads. The fusion proteins are eluted by incubating the beads in a solution of 5 mM reduced glutathione, 50 mM Tris HCl (pH 8.0). The eluted recombinant proteins are concentrated and dialysed in 0.1 M phosphate buffer pH 7.4. The quality of the purified proteins was checked on SDS-PAGE gel and the concentration of the proteins determined using the Bradford test (Bio-Rad kit).

The native genes of the GRA5 protein derived from different strains were cloned into a pGEX 3X vector containing the cleavage site for factor Xa between the GST and the protein of interest.

The polypeptide of SEQ ID NO.3 (fragment of GRA5-RH, without the C-terminal threonine) fused with GST has the following sequence:

SEQ ID NO. 13:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G R↓G
I P G G S T R D V G S G G D D S E G A R G R E Q Q
Q V Q Q H E Q N E D R S L F E R G R A A V T G H P
V R E F I V T D

The GST sequence appears before the cleavage site (↓), between the Factor Xa recognition site, shown in bold underlined type, and the polypeptide derived from the GRA5-RH protein (in bold type), itself flanked by amino acids created by the polylinker of the insertion site, shown in italics.

The polypeptide of SEQ ID NO.5 (fragment of GRA5-76K, without the C-terminal threonine) fused with GST has the following sequence:

SEQ ID NO. 14:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G R↓G
I P G G S T C D T G S G G D D S E G A W G G E Q Q
Q V Q Q H G Q S E D R S L F E R G R A A V T G H P
V R E F I V T D

The polypeptide of SEQ ID NO.6 (fragment of GRA5-CEP or GRA-056, without the C-terminal threonine) fused with GST has the following sequence:

SEQ ID NO. 15:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G R↓G
I P G G S T R D V G S G A D D S E G A G G R E R Q
Q V Q Q H E Q N E D R S L F E R G R A A V T G H P
V R E F I V T D

### Analysis of the Polymorphism of the GRA5 Genes (RH, 76K, Pru, CEP and C56 Strains)

The oligonucleotide primer pair (21.19 and 21.20) was used to amplify the fragments of genomic DNA corresponding to the GRA5 gene by PCR (Lecordier et al., Mol. Biochem. Parasitol. 1993, 59, 143-154) using DNA from each parasite strain. The amplified fragments contain the untranslated 5′ region (5′ UT), the open reading frame and the untranslated 3′ region (3′ UT) of the GRA5 gene. The size of the amplified fragments (˜0.95 kb) was checked on a 1% agarose gel. After purification, the fragments were cloned into a PCR-Script SK(+) vector digested with SfrI. The cloned fragments were sequenced in both directions. The sequences obtained were aligned with the known sequence of the European RH strain of genotype I (Lecordier et al. 1993, cited above; GenBank accession number: L06091) (FIGS. 1 and 2). On FIG. 2, the deduced amino acid sequences of the GRA5 gene obtained in the 76K and Pru strains for group II and in the CEP and C56 strains for group III were aligned with that of the group I RH strain. The amino acids that are identical to those of the RH strain which was adopted as the reference were labelled “−”. The substituted amino acids are indicated, and non-conservative substitutions are indicated in bold type. The amino acids corresponding to the two hydrophobic regions (signal peptide and transmembrane domain) that flank the amino acids of the N-terminal region which was tested, are indicated in italics.

A comparative analysis of the coding sequences of GRA5 obtained from the strains of the three types (I, II, III) revealed:
1. that the sequence within each group is very highly conserved, with 99.72% homology between the 76K and Pru strains of group II and 100% homology between the CEP and C56 strains of group III;
2. greater homology between the strains of groups I and III (greater than 99%) than between the strains of groups I and II (approximately 98%);
3. all the substitutions, except for the final guanine of the strains of group II, alter the primary protein sequence, with six different amino acids in the Pru strain (group II) and three in the group III strains (FIG. 2);
4. the presence of a polymorphic region within the N-terminal region, after the potential cleavage site of the signal peptide (sic) most of the substituted amino acids is present in the N-terminal region of the protein, with the exception of one substitution in the strains of group II, where a lysine (K94) was replaced by an arginine (R);
5. that of the six substitutions observed in the sequence of the type II GRA5 proteins, three are conservative and three others pertain to charged amino acids being replaced by hydrophobic amino acids in the type II sequence (R42/W42; R44/G44; E53/G53);
6. that of the three substitutions observed in the sequence of the type III GRA5 proteins, two are conservative, and the third also corresponds to replacement of a charged amino acid by a hydrophobic residue (R42/G42).
   Expression and Purification of GRA5 Variants (Hydrophilic N-Terminal Region) in the Form of a Recombinant Polypeptide Fused with GST.

The type I, II and III variants of the hydrophilic N-terminal region of GRA5 were expressed in the form of recombinant polypeptides fused to glutathione S-transferase (GST) using the expression vector pGEX-3X. To achieve this, the expression plasmids pTg GRA5-Nt type I, pTg GRA5-Nt type II and pTg GRA5-Nt type III were constructed. The DNA sequences used for the expression of the variants GRA5 Nt types I, II and III are those of GRA5 RH, 76K CEP and C56 (FIG. 1). The 150 base-pair (bp) fragment encoding the N-terminal portion of GRA5 was amplified using the G5N5′ (or G5N5′76K) and G5N3′ primer pair. The amplified fragments were digested with SmaI and EcoRI and inserted by ligation into the SmaI/EcoRI sites of the pGEX 3X vector, in phase with the sequence encoding glutathione S-transferase (GST). The constructs were checked by sequencing.

For the expression of the recombinant proteins, competent BL 21 cells were transformed with parental plasmid DNA (pGEX-3X) or recombinant plasmid (GRA5-Nt type I, pTg GRA5-Nt type II, pTg GRA5-Nt type III). After induction of gene expression followed by purification, the fusion proteins were analysed on SDS-PAGE gel. The yield for GST production is 25 mg per litre of culture and 40 to 50 mg per litre of culture for the three fusion proteins (GST-GRA5-Nt type I, GST-GRA5-Nt type II, GST-GRA5-Nt type III).

### In Vitro Assay of Dendritic Cell Migration

Culture of Dendritic Cells and Langerhans Cells

Dendritic cells are derived by differentiation from CD34 precursor cells isolated from umbilical cord blood, as indicated in the following references: Rougier et al., J. Invest. Dermatol. 1998, 110, pp 348-352 and Eur. J. Cell. Biol. 1998, 75 pp 287-293. After 4 to 6 days of differentiation, the cells obtained are frozen in liquid nitrogen and can be stored for several years.

Migration Assay

The assay used is described in the two publications Staquet et al. (Int Arch Allergy Immunol. 2004, 133, 348-56), and Diana et al. 2004 cited above. Plates with or without Matrigel can be used, with comparable results.
1. The dendritic cells are thawed and cultured in RPMI medium with 5% FCS, gentamicin, GM-CSF and TNFα to obtain interstitial dendritic cells. Medium containing GM-CSF and TGFβ1 is used in order to obtain Langerhans cells. The two types of cell function in an equivalent manner in the migration assay.
2. The cells are counted 48 hours later and 300,000 cells are placed in each Falcon culture tube in a volume of 300 μl. The following activators are added: Bandrowski's base (at a final concentration of 1.27 ng/ml), LPS (at a final concentration of 25 ng/ml) or ESAs at a final dilution of 1:20. The tubes are returned to culture.
3. If migration is going to be performed in the presence of Matrigel, the Matrigel inserts are treated as follows: add 735 μl of RPMI medium to the tube of Matrigel removed from the freezer and thaw rapidly by pipetting up and down. Next add an equal volume of RPMI medium and mix gently to avoid bubble formation; the Matrigel concentration is 0.5 mg/ml. These 2 ml of solution are sufficient to prepare 4 inserts for a 6-well plate, 19 inserts for a 12-well plate, 66 inserts for a 24-well plate.
4. Position the inserts in their respective plates. The gel is applied gently, using a micropipette, avoiding bubble formation. Apply 30 μl onto each 24-well plate insert, 100 μl onto each 12-well plate insert and 420 μl onto each 6-well plate insert. Allow to evaporate under the hood for between 4 and 20 hours. Rinse each insert twice with RPMI before applying the cell suspension.
5. After 24 hours, the tubes are recovered, and the cells are counted and resuspended in the culture medium with 1% bovine serum albumin.
6. A constant number of cells is loaded into each insert, and medium containing MIP-3β is added to the wells. The quantity of cells and the volumes used depend on the type of insert and are indicated in the following table:

| Type of | dendritic | | |
|---|---|---|---|
| plate | cells/insert | Volume/insert | Volume/well |

| | | | | | |
|---|---|---|---|---|---|
| X 6 | 300,000 | 1 | ml | 2 | ml |
| X 12 | 150,000 | 0.4 | ml | 1 | ml |
| X 24 | 50,000 | 0.2 | ml | 0.4 | ml |

7. After 24 hours, the inserts are removed and the cells that crossed them and are at the bottom of the wells are recovered and counted. The results are expressed as the percentage of cells relative to the number originally placed in the insert.

### Results

1. The Supernatant Obtained from Culturing Tachyzoites Contains an Activity That Induces the Migration of Dendritic Cells.

The results presented in FIG. 3 correspond to between 6 and 20 experiments, depending on the fraction. They are normalised by calculating a migration index with reference to the untreated control (C). The positive controls BB and LPS correspond to dendritic cells activated by Bandrowski's base (a chemical allergen) and by LPS (a molecule of bacterial origin). The standard deviation was calculated when more than three different experiments were performed.

The supernatant from cultured tachyzoites of the RH strain (ESA-RH) and that of the PRU strain (ESA-PRU) induce the migration of dendritic cells as indicated in the figure.

A soluble extract of PRU tachyzoites (EX-PRU) obtained by 3 cycles of freeze-thawing then elimination of the insoluble molecules by centrifugation is inactive. A culture supernatant from cells of the human cell line THP1, which was used to produce the tachyzoites in vitro, is also inactive (SUP-THP1).

2. The Activity is Controlled by the GRA5 Gene

FIG. 4 shows the results obtained by inducing migration with supernatants from the RH and RH HX-strains, which both induce migration in 11 different experiments. The HX-RH strain was used to produce three strains in which the GRA2, GRA5 or GRA6 genes were respectively disrupted (Mercier et al., 2001, cited above). Only the GRA2- and GRA6-supernatants induce migration that is significantly different from the control, and the strain in which the GRA5 gene is disrupted no longer produces the activity. Therefore, the secretion products of the GRA5 mutant strain have lost their capacity to induce the migration of dendritic cells.

The GRA5-RH gene is reintroduced along with the GST gene into the GRA5-knockout strain (Mercier et al., 2001, above) and the new strain (labelled GRA5−/+) produces active supernatant. The previous results were reproduced in 5 different experiments. These data implicate the GRA5 gene in the migration-inducing activity.

3. The Activity Resides in the N-Terminal Portion of the Molecule

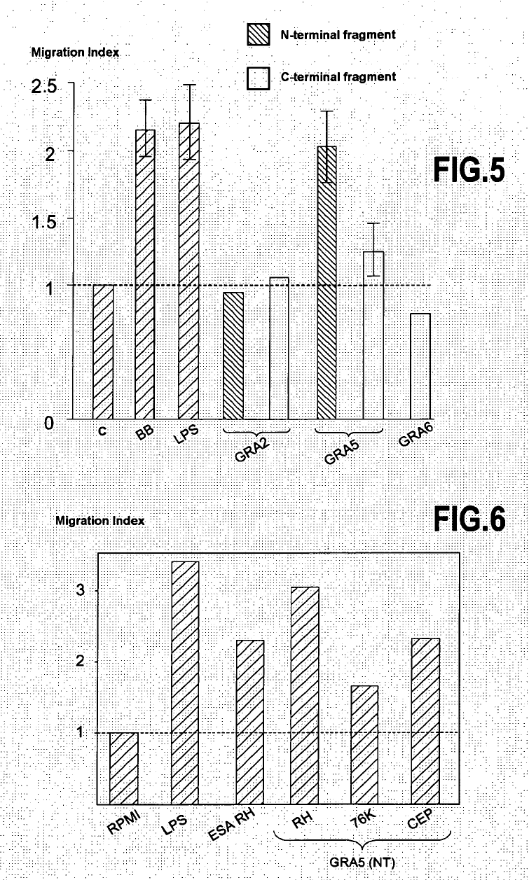

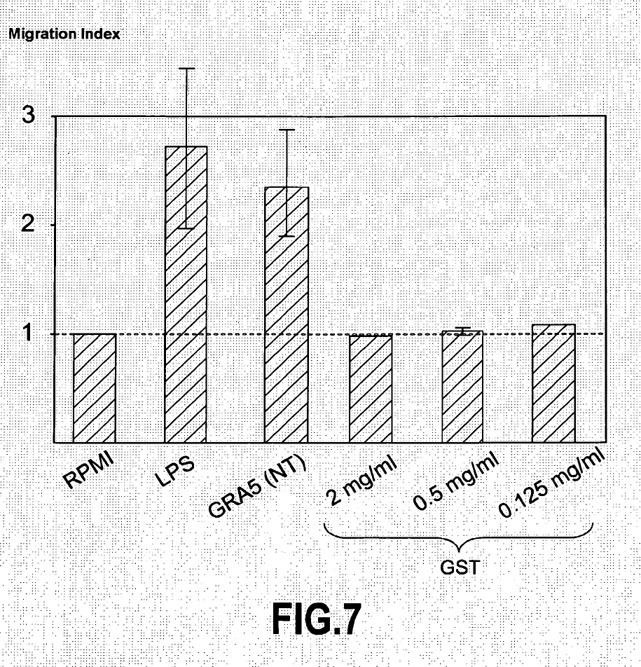

GST fusion proteins incorporating the N-terminal portion of the GRA5 protein prepared previously (SEQ ID NO.13, 14 and 15 for the N-terminal portion) were introduced into the dendritic cell migration assay. Similarly, GST fusion proteins using the C-terminal portion of the GRA5 protein, were produced and tested, as well as fusion proteins with the C- and N-terminal portions of the GRA2 and GRA6 proteins. FIG. 5 gives the migration indexes obtained with these fusion proteins (labelled GRA5, GRA2 and GRA6 on the figure) for the RH strain. As indicated on FIG. 5, only the N-terminal part of GRA5 possesses the activity. FIG. 6 illustrates the migration indexes obtained with the fusion proteins of SEQ ID NO.13, 14 and 15 corresponding to strains RH, 76K and CEP, respectively. It was further demonstrated that the activity does not reside in the GST sequence added to the various fragments to facilitate their production in a bacterial system and their purification, for two reasons: the GST molecule itself has no activity as shown in FIG. 7, and finally the C-terminal fragment of GRA5 has no activity, as is the case for the GRA2 and GRA6 fragments (FIG. 5). Another finding indicating that the activity resides in the N-terminal portion of GRA5 is the fact that the fragments from different strains have variable activities, as indicated in FIG. 6.

### Sequence of the Proteins of Interest

Sequence of the Whole GRA5 Protein from the RH Strain

SEQ ID NO. 1

MASVKRVVVA VMIVNVLALI FVGVAGSTRD VGSGGDDSEG
ARGREQQQVQ QHEQNEDRSL FERGRAAVTG HPVRTAVGLA
AAVVAVVSLL RLLKRRRRRA IQEESKESAT AEEEEVAEEE

### Sequence of the N-Terminal Active Fragment

RH STRAIN

SEQ ID NO. 3

GSTRDVGSGG DDSEGARGRE QQQVQQHEQN EDRSLFERGR
AAVTGHPVRT
PRU STRAIN

SEQ ID NO. 4

GSTRDTGSGG DDSEGAWGGE QQQVQQHGQS EDRSLFERGR
AAVTGHPVRT
76K STRAIN

SEQ ID NO. 5

GSTCDTGSGG DDSEGAWGGE QQQVQQHGQS EDRSLFERGR
AAVTGHPVRT
C56 AND CEP STRAIN

SEQ ID NO. 6

GSTRDVGSGA DDSEGAGGRE RQQVQQHEQN EDRSLFERGR
AAVTGHPVRT

Various assays were performed with each of these polypeptides and show that these polypeptides cause the migration of dendritic cells and Langerhans cells. Immunolabeling of the cells showed that the addition of each of these polypeptides:
1. causes a decrease in E-cadherin on the membrane (a molecule involved in intercellular interactions in the epidermis).
2. causes loss of CCR6, the receptor for the chemokine MIP-3α, produced in the epidermis, and that stabilises dendritic cells at this site.
3. causes the appearance of CCR7, the receptor for the chemokine MIP-3β, produced in lymph nodes, and that attracts dendritic cells away from the epidermis.
4. must cause the synthesis of metalloproteases, the latter result being deduced from the fact that the dendritic cells migrate even when the inserts are treated with Matrigel. Matrigel has the composition of a dermal-epidermal basement membrane and in vivo dendritic cells must synthesise metalloproteases in order to cross it (Vincent et al. Eur. J. Cell. Biol. 2002, 81, 383-389).
   Together these findings show that the dendritic cells have the capacity to dissociate from their environment and cross the basement membrane to move towards the draining lymph nodes.

The Activity of the GRA5-PRU Fragments

The polypeptides with sequences:

| | |
|---|---|
| SEQ ID NO. 16: | DTGSGGDDSEGAWGGEQQQVQQHGQSEDR |
| SEQ ID NO. 17: | DSEGAWGGEQQQVQQHGQSEDRSLFER |

were prepared by peptide synthesis.

These peptides were then subjected to the dendritic cell migration assay described previously. Dendritic cells generated in vitro from precursor cells isolated from cord blood were incubated overnight in the presence of decreasing doses of the peptide and the next day their ability to migrate towards the chemokine MIP-3β was tested. FIG. 8 shows the in vitro migration-inducing activities of the peptide of SEQ ID NO:16 and of the peptide of SEQ ID NO.17 (each point represents the mean migration observed in 5 experiments) at different concentrations.

The peptide of SEQ ID NO.16 was also tested in vivo in mice in a migration-inducing assay. In order to do this, a population of mice was used in which the EGFP gene is introduced in front of the gene for Langerin, which makes Langerhans cells naturally fluorescent. The gene encoding CCR6 is also disrupted in these mice but normal numbers of Langerhans cells are present in the epidermis. These mice are obtained by crossing two murine strains: the Langerin DTR EGFP line (Kissenpfennig A, et al. in Trends Immunol. 2006 March; 27(3):132-9. Immunity. 2005 May; 22(5):643-54) maintained at the CNRS centre for distribution, typing and archiving at Orleans, France, and subsequently available from the European Mouse Mutant Archive (EMMA, GSF National Research Centre for Environment and Health, Institute of Experimental Genetics, Ingolstädter Landstrasse 1, D-85764 Neuherberg, Munich, Germany, www.emmanet.org) under the identification number EM:01822, and the CCR6 knockout line, published by Cook et al. in Immunity, 2000, 12: 495-503 and by Lukrs et al. in J. Exp. Med., 2001, 194, 551-555.

The peptide of SEQ ID NO.16 is prepared in DMSO at a concentration of 25 mg/ml. 5 μl of this preparation is applied to the left ear of the mouse and an equivalent volume to the right ear. The mouse is sacrificed 24 hours later and the auricular lymph nodes removed. The number of (naturally fluorescent) Langerhans cells is counted and the total number of dendritic cells identified by labelling with an anti-CD11c antibody.

A greater number of dendritic cells and Langerhans cells is observed in the lymph nodes on the left side than on the right side, which indicates that the peptide of SEQ ID NO.16 induces the migration of these cells in vivo. FIG. 9 shows the number of Langerhans cells and dendritic cells (identified by CD11c antibody labelling) obtained on the side treated with the peptide of SEQ ID NO.16 and on the side treated with DMSO alone.

1. Polypeptide corresponding to amino acids 26 to 75 of the sequence of the GRA5-RH protein of
SEQ ID NO. 1:
MASVKRVVVAVMIVNVLALIFVGVAGSTRDVGSGGDDSEGARGRE
QQQVQQHEQNEDRSLFERGRAAVTGHPVRTAVGLAAAVVAVVSLL
RLLKRRRRRAIQEESKESATAEEEEVAEEE
variants thereof with activity on the migration of dendritic cells, homologues thereof with activity on the migration of dendritic cells, and fragments thereof with activity on the migration of dendritic cells.

2. Polypeptides according to claim 1 selected from the polypeptides of sequence:
SEQ ID NO. 3
GSTRDVGSGG DDSEGARGRE QQQVQQHEQN EDRSLFERGR
AAVTGHPVRT
SEQ ID NO. 4
GSTRDTGSGG DDSEGAWGGE QQQVQQHGQS EDRSLFERGR
AAVTGHPVRT
SEQ ID NO. 5
GSTCDTGSGG DDSEGAWGGE QQQVQQHGQS EDRSLFERGR
AAVTGHPVRT
SEQ ID NO. 6
GSTRDVGSGA DDSEGAGGRE RQQVQQHEQN EDRSLFERGR
AAVTGHPVRT
homologues or fragments thereof, with activity on the migration of dendritic cells.

3. Polypeptides according to claim 1 selected from the polypeptides of sequence:
SEQ ID NO. 3
GSTRDVGSGG DDSEGARGRE QQQVQQHEQN EDRSLFERGR
AAVTGHPVRT
SEQ ID NO. 4
GSTRDTGSGG DDSEGAWGGE QQQVQQHGQS EDRSLFERGR
AAVTGHPVRT
SEQ ID NO. 5
GSTCDTGSGG DDSEGAWGGE QQQVQQHGQS EDRSLFERGR
AAVTGHPVRT
SEQ ID NO. 6
GSTRDVGSGA DDSEGAGGRE RQQVQQHEQN EDRSLFERGR
AAVTGHPVRT

4. Polypeptides according to claim 1 selected from the fragments corresponding to amino acids 30 to 58, or 37 to 63 of the sequence of a GRA5 protein, and in particular of the sequence of a GRA5 protein from the strains RH, PRU, 76K, CR6 or CEP, homologues thereof with activity on the migration of dendritic cells, as well as fragments thereof with activity on the migration of dendritic cells.

5. Polypeptides according to claim 4 selected from the polypeptides of sequence:

| | |
|---|---|
| SEQ ID NO. 16: | DTGSGGDDSEGAWGGEQQQVQQHGQSEDR |
| SEQ ID NO. 17: | DSEGAWGGEQQQVQQHGQSEDRSLFER |

homologues thereof with activity on the migration of dendritic cells, as well as fragments thereof with activity on the migration of dendritic cells.

6. Purified or isolated nucleic acid consisting of a sequence of nucleotides encoding a polypeptide according to claim 1.

7. Fusion protein between GST and a polypeptide according to claim 1 as well as the homologues or fragments of such proteins, with activity on the migration of dendritic cells.

8. Protein according to claim 7 selected from:
the protein of SEQ ID NO. 13:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G R G
I P G G S T R D V G S G G D D S E G A R G R E Q Q
Q V Q Q H E Q N E D R S L F E R G R A A V T G H P
V R E F I V T D
the protein of SEQ ID NO. 14:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G R G
I P G G S T C D T G S G G D D S E G A W G G E Q Q
Q V Q Q H G Q S E D R S L F E R G R A A V T G H P
V R E F I V T D
the protein of SEQ ID NO. 15:
S P I L G Y W K I K G L V Q P T R L L L E Y L E E
K Y E E H L Y E R D E G D K W R N K K F E L G L E
F P N L P Y Y I D G D V K L T Q S M A I I R Y I A
D K H N M L G G C P K E R A E I S M L E G A V L D
I R Y G V S R I A Y S K D F E T L K V D F L S K L
P E M L K M F E D R L C H K T Y L N G D H V T H P
D F M L Y D A L D V V L Y M D P M C L D A F P K L
V C F K K R I E A I P Q I D K Y L K S S K Y I A W
P L Q G W Q A T F G G G D H P P K S D L I E G R G
I P G G S T R D V G S G A D D S E G A G G R E R Q
Q V Q Q H E Q N E D R S L F E R G R A A V T G H P
V R E F I V T D
as well as the homologues or fragments of such proteins, with activity on the migration of dendritic cells.

9. Polypeptides according to claim 1, as medicinal products.

10. Pharmaceutical compositions, particularly those suitable for topical administration, containing a polypeptide according to claim 1, in combination with at least one pharmaceutically suitable excipient.

11. The use of a polypeptide according to claim 1, for the manufacture of a medicinal product intended for the prevention or treatment of diseases of the skin or mucosae in which dendritic cells or Langerhans cells are implicated.

12. The use of a polypeptide according to claim 1, for the manufacture of a medicinal product intended for the prevention or treatment of diseases selected from: atopic dermatitis, inflammations, autoimmune diseases, systemic lupus erythematosus, polyarthritis, psoriasis, graft versus host disease, graft rejection, or allergies, such as contact allergies, for example to latex, cosmetics, perfumes, tattoos, metals and their salts, pulmonary allergic phenomena due to airborne allergens, asthma, allergic rhinitis or food allergies.

13. Pharmaceutical compositions, particularly those suitable for topical administration, containing a fusion protein according to claim 7, in combination with at least one pharmaceutically suitable excipient.

14. The use of a fusion protein according to claim 7, for the manufacture of a medicinal product intended for the prevention or treatment of diseases of the skin or mucosae in which dendritic cells or Langerhans cells are implicated.

15. The use of a fusion protein according to claim 7, for the manufacture of a medicinal product intended for the prevention or treatment of diseases selected from: atopic dermatitis, inflammations, autoimmune diseases, systemic lupus erythematosus, polyarthritis, psoriasis, graft versus host disease, graft rejection, or allergies, such as contact allergies, for example to latex, cosmetics, perfumes, tattoos, metals and their salts, pulmonary allergic phenomena due to airborne allergens, asthma, allergic rhinitis or food allergies.
